(19) United States
(12) Patent Application Publication
(10) Pub. No.: US 2012/0048277 A1
(43) Pub. Date: Mar. 1, 2012

(54) TRACHEAL TUBE WITH ADJUSTABLE FLANGES

(75) Inventors: Paul Waldron, Galway (IE); Seamus Maguire, Athlone (IE); Roger Harrington, Athlone (IE); Aaron Macan, Loveland, CO (US); Mark R. Behlmaier, Erie, CO (US)

(73) Assignee: Nellcor Puritan Bennett LLC, Boulder, CO (US)

(21) Appl. No.: 12/873,484

(22) Filed: Sep. 1, 2010

(51) Int. Cl. A61M 16/04

(52) U.S. Cl. 128/207.14

(57) ABSTRACT

Systems and methods that utilize tracheal tubes having one or more adjustable flanges are described. The adjustable flanges may be disposed on the tracheal tube at various angles. Further, the adjustable flanges may be repositioned on the tracheal tube, thus allowing a clinician or patient to reconfigure the tracheal tube in situ so as to more comformably fit the tracheal tube to a variety of patient anatomies. Flange extensions are also provided that may extend the overall flange length, and may also be repositioned at various angles. The use of techniques such as adjustable flanges and adjustable flange extensions allow a better fit to a particular patient's anatomy and provide increased patient comfort.

### BACKGROUND

The present disclosure relates generally to tracheal tubes and, more particularly, to tracheal tubes having adjustable flanges.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Tracheal tubes may be utilized to define a clear passageway for air, other gases, and medicaments to the trachea and lungs, thus providing an artificial airway for spontaneous or mechanical ventilation of a patient. Such tracheal tubes may include endotracheal tubes and tracheostomy tubes. Tracheostomy tubes, for example, are typically introduced into an opening or stoma in front of the neck and trachea. The stoma is defined by a tracheotomy incision in the neck that provides access to the trachea. When the inner or distal end of the tracheostomy tube is properly inserted into the trachea, the tracheostomy tube may be secured in place by flanges (e.g., “wings”). The flanges may be positioned on the neck and may then be secured in placed by a strap such as a ribbon or soft tie. However, in patients having certain neck obstructions and/or having unusual anatomies, the flanges may not be suitable for their intended purpose.

### BRIEF DESCRIPTION OF THE DRAWINGS

Advantages of the disclosed techniques may become apparent upon reading the following detailed description and upon reference to the drawings in which:

FIG. 1 illustrates a first embodiment of a tracheal tube having adjustable flanges;

FIG. 2 is an exploded view of an embodiment of the tracheal tube of FIG. 1 having adjustable flanges;

FIG. 3 is a cross-section view of an embodiment of the tracheal tube of FIG. 1;

FIG. 4 illustrates a second embodiment of a tracheal tube having adjustable flanges;

FIG. 5 is an exploded view of an embodiment of the tracheal tube of FIG. 4;

FIG. 6 is a perspective view of an embodiment of the tracheal tube of FIG. 4;

FIG. 7 is a perspective view of a third embodiment of a tracheal tube having adjustable flanges;

FIG. 8 is a perspective view of an embodiment of the tracheal tube of FIG. 7 with an end connector removed;

FIG. 9 is an exploded view of an embodiment of the tracheal tube of FIG. 7;

FIG. 10 is a perspective view of a fourth embodiment of a tracheal tube having adjustable flanges;

FIG. 11 is an exploded view of an embodiment of the tracheal tube of FIG. 10;

FIG. 12A is a frontal view of embodiments of components of the tracheal tube of FIG. 10;

FIG. 12B is another frontal view of embodiments of components of the tracheal tube of FIG. 10;

FIG. 13 is an embodiment of a tracheal tube having adjustable flange extensions;

FIG. 14 is an exploded view of an embodiment of the tracheal tube of FIG. 13;

FIG. 15 is another embodiment of a tracheal tube having adjustable flange extensions;

FIG. 16 is an exploded view of an embodiment of the tracheal tube of FIG. 15;

FIG. 17 is a perspective view of an embodiment of a tracheal tube having adjustable flanges;

FIG. 18 is a side view of an embodiment of the tracheal tube of FIG. 17; and

FIG. 19 is an exploded view of an embodiment of the tracheal tube of FIG. 17.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The disclosed embodiments include medical devices for artificial airway applications. In certain embodiments, a tracheal tube, such as a tracheostomy tube, is provided that includes one or two adjustable flanges. The tracheostomy tube may be inserted into the trachea via a surgical incision in the neck. After insertion of the tube into the trachea, a portion of the tracheal tube, i.e., the proximal portion, remains outside the patient while a distal portion is positioned inside the trachea. Accordingly, the tracheal tube provides for a fluid conduit into the patient's airway. The proximal portion extends outwardly from the neck and may include a proximal end connector, such as a 15 mm outer diameter (OD) end connector, a 8.5 mm end connector, or any other suitably-sized end connector capable of connecting the tracheostomy tube to a ventilator, suctioning equipment, or other medical device. The proximal portion of the tube may be secured in place on the patient's neck by using adjustable flanges that rest on the neck and are further secured by straps circumferentially encircling the neck. The adjustable flanges enable a user such as a caregiver or a patient, to more quickly and securely attach the tracheal tube, such as a tracheostomy tube, to a wide range of neck anatomies. Further, the adjustable flanges may decrease patient discomfort during regular use by more comformably securing the tracheal tube. That is, the adjustable flanges may be custom fit to a specific patient's neck area so as to better adapt to the patient's anatomy and/or any obstruction in the neck. Accordingly, patients having larger neck diameters or having other medical devices positioned in the neck area, may be more suitably accommodated by using the techniques disclosed herein.

In certain embodiments, each adjustable flange may rotate 360° radially about the proximal end connector. Accordingly, the tracheal tube may be comformably fit to any number of angles, as desired. In other embodiments, the adjustable flanges may rotate less than 360°. In one example, the adjustable flanges may symmetrically rotate with respect to each other. That is, if a first flange rotates a certain number of degrees, such as 15°, with respect to an axis, a second flange will also rotate the same number of degrees (e.g., 15°) with respect to the same axis. Symmetric movement of the flanges may enable a more secure neck attachment because the neck straps securing the flange are more likely to meet at the same angle, creating an approximately circular attachment around the neck. In other embodiments, the first flange may be positioned at a different angle compared to the second flange. Such asymmetrical positioning may enable the flanges to overcome obstructions in the neck area such as a catheter (e.g., central venous catheter), sutures, a medical instrument (e.g., endoscope), anatomical structures, and so forth.

In some examples, the flanges may be first positioned at a certain angle and then “locked” in place. In these examples, a variety of locking and unlocking mechanisms may be used, including flexible rings, buttons, engageable protrusions (e.g., “teeth”), and so forth. In certain embodiments, the flanges may also incorporate adjustable flange extensions. Such flange extensions may extend the length of the flanges and may also be adjusted at any number of angles, as desired. By incorporating adjustable flanges and adjustable flange extensions, a tracheal tube may be adjusted to more comformably fit to a wide variety of neck anatomies. Indeed, the adjustable flanges may allow for a proper positioning of the tracheal tube in a wide range of patients, including pediatric and adult patients.

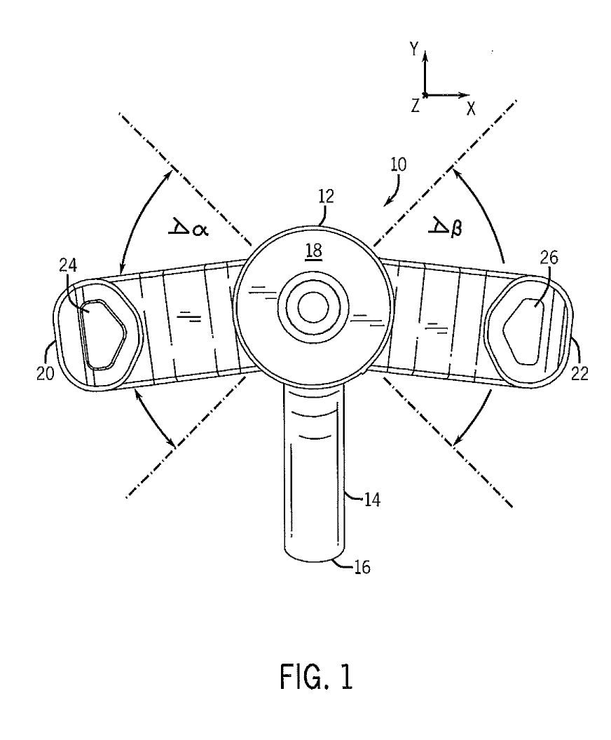

With the foregoing in mind, FIG. 1 depicts an embodiment of tracheal tube 10 that may be utilized to provide respiratory support in a patient. A tracheostomy incision is typically made in the patient trachea and a tracheal tube 10 is inserted into the trachea. The tracheal tube 10 includes a proximal end portion 12 and a distal end portion 14. The distal end portion 14 is inserted into the trachea and typically includes a curved portion so as to comformably fit inside the patient airway. In certain embodiments, the outer diameter (OD) of the distal end portion 14 may be approximately 1 mm-20 mm, which may vary depending on whether the patient is a neonatal patient, a pediatric patient or an adult patient. The distal end portion 14 may be any suitable length. For example, the distal end portion 14 may be 50 mm-175 mm. A distal opening 16 may be beveled to allow for smoother insertion through the larynx and trachea. The tracheal tube 10 may also include any suitable number of lumens that may be appropriately sized and shaped for inflation, deflation, suction, or drug delivery for example.

The tracheal tube 10 may also include an end connector 18 that may be used to couple a variety of medical devices, such as ventilator, a manual respirator, suctioning equipment, and so forth, to the tracheal tube 10. In certain embodiments, the proximal end connector 18 may include a 15 mm OD portion, i.e., male end connector that can couple with a standard 15 mm inner diameter (ID) connector, i.e., female end connector. It is to be understood that in other embodiments, the proximal end connector 18 may include a male connector portion of a different size, including international organization for standardization (ISO) sizes such as 8.5 mm OD, 22 mm OD, 23 mm OD, 30 mm OD and so on. Indeed, all tracheal tube embodiments described herein may include proximal end connectors having the aforementioned ISO sizes.

FIG. 1 also illustrates an embodiment of angularly adjustable flanges 20 and 22. The adjustable flanges 20 and 22 rest on the patient's neck and may be further secured by straps to the patient. Accordingly, flange openings 24, 26 enable the use of straps to be placed circumferentially around the neck and attached to the flange openings 24 and 26, thus securing the tracheal tube 10 to the patient. A stable attachment of the tracheal tube 10 to the patient is advantageous for preventing motion of the tube within the trachea. Accordingly, the flanges 20, and 22 may be positioned radially about the end connector 18 so as to enable a more secure attachment to the patient. In the depicted embodiment, the user (e.g., caregiver or patient) may adjust flange 20 so as to position the flange 20 at a certain angle α (on the X-Y plane) suitable for a more secure attachment to the patient. Likewise, the user may also adjust flange 22 so as to position the flange 22 at a certain angle β (on the X-Y plane). Indeed, both flanges 20 and 22 may be radially moved about the Z-axis at any of a plurality of angles such that both flanges 20 and 22 may be more securely and comformably attached to the patient's neck. In another embodiment, one of the flanges 20 or 22 may be angularly adjustable while the other flange 20 or 22 may be non-angularly adjustable (i.e., “fixed”). That is, one flange, such as the flange 20, may be repositionable at a number of desired angles relative to the end connector 18 while the other flange, such as the flange 22 may be manufactured to remain at a fixed angle. Indeed, it is to be understood that all of the tracheal tube examples disclosed herein, such as in FIGS. 1-19, may include embodiments having one adjustable flange and one non-adjustable flange. Such embodiment may limit the number of interstices (e.g., spaces or gaps) included in the tracheal tube embodiments, thus reducing the locations that may harbor bacteria.

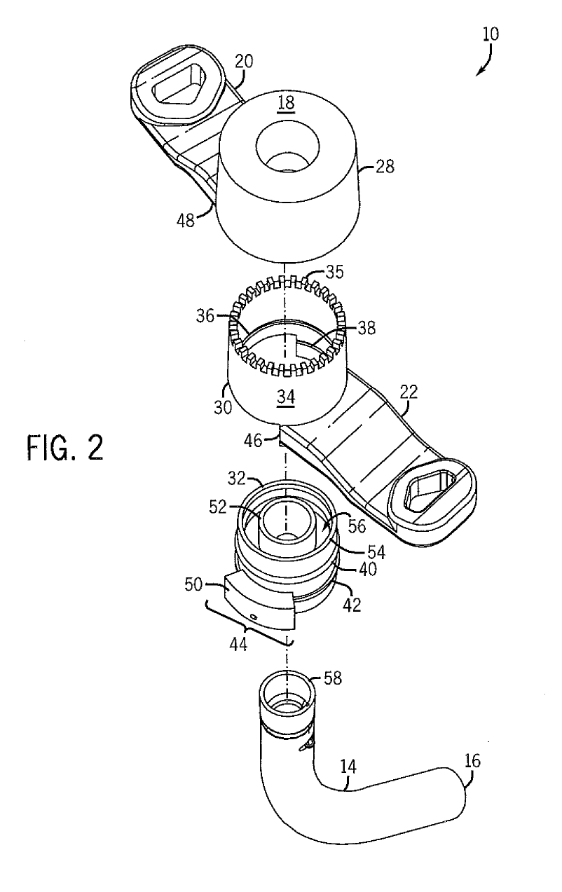

In certain cases, such as with a patient having a large diameter neck, the tracheal tube 10 may more securely be attached to the patient by adjusting the flanges 20 and 22 so that both flanges are at approximately 90° with respect to the distal end portion 14 and thus the length of the distance between flange openings 24 and 26 is maximized. That is, for larger neck anatomies, the flanges 20, 22 and the proximal end portion 14 may form a “T” shape, with the horizontal portion of the “T” formed by the flanges 20 and 22 and the vertical portion of the “T” formed by the distal end portion 14. Accordingly, a larger area of the patient's neck may be covered by the flanges 20 and 22. In patient's having a smaller neck diameter, the flanges 20 and 22 may be positioned so as to reduce the distance between the openings 24 and 26. For example, the flanges 20, 22 and the proximal end portion 14 may form a “Y” shape, with the angled arms of the “Y” formed by the flanges 20 and 22 and the vertical portion of the “Y” formed by the distal end portion 14. Indeed, the flanges 20 and 22 may be rapidly positioned to any number angles, including angles that result in an asymmetrical positioning of the flanges 20 and 22 (i.e., angles where α≠β). Once the flanges 20, 22 have been positioned, certain techniques, such as the techniques described in more detail with respect to FIG. 2, may be used to lock the flanges 20 and 22 at the desired angles.

FIG. 2 is an exploded view of embodiments of components of the tracheal tube 10 that may include techniques, such as engageable teeth, capable of locking the two flanges 20 and 22 at various angles. The figure is also illustrative of how the various components of the tracheal tube 10 may be assembled or manufactured. The figure shows four components, 28, 30, 32, and 14. The component 28 may include the end connector 18 and the flange 20. The component 30 may include a barrel 34 and the flange 22. A core 32 suitable for coupling with the distal end portion (i.e., cannula) 14 may also be included as another component of the tracheal tube 10. The components 28, 30, 32 and 14 may be molded, cast, milled, and so forth. Some example materials that may be used in the manufacturing of the tracheal tube 10 components 28, 30, 32 and 14 may include acrylonitrile butadiene styrene (ABS), phthalate-free polyvinyl chloride (PVC), polyethylene terephthalate (PET), low-density polyethylene (LDPE), polypropylene, silicone, neoprene, and/or polyisoprene. Indeed, it is to be understood that all of the tracheal tube embodiments disclosed herein, such as in FIGS. 1-19, include components that may be molded, cast, and/or milled out of materials such as ABS, PVC, PET, LDPE polypropylene, silicone, neoprene, and/or polyisoprene.

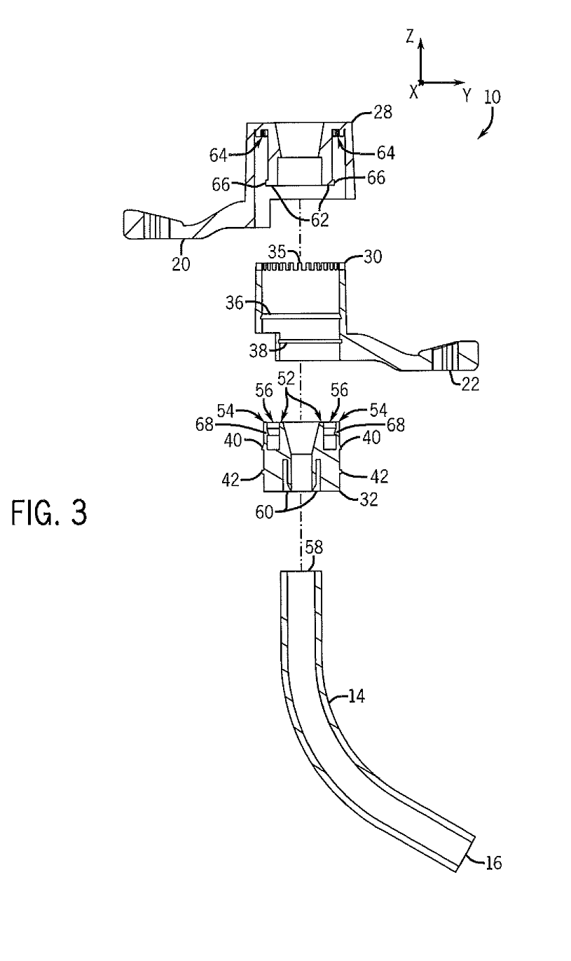

The tracheal tube 10 may be manufactured or assembled by axially inserting the barrel 34 of the component 30 into the rear end of the end connector 18 of the component 28. In this embodiment, the rear end of the end connector 18 includes a hollow channel manufactured at a dimension suitable for accepting the barrel 34, as described in more detail below with respect to FIG. 3. Accordingly, the barrel 34 may include an OD smaller than the ID of the end connector 18. The barrel 34 may also include a plurality of square protrusions (i.e., “teeth”) 35 that can engage a corresponding set of square notches positioned in the rear of the end connector 18. It is to be understood that in other embodiments, other shapes for the protrusions 35 may be used that are also capable of engaging a corresponding notch, such as triangle shapes, and/or curved shapes. Once the square teeth 35 engage the corresponding notches (i.e., are positioned inside of the corresponding notches), the square teeth 35 and corresponding notches may then “lock” the components 28 and 30 together. This locking prevents the radial movement of the flanges 20 and 22 with respect to the end connector 28. That is, the flanges 20 and 22 may not “turn” with respect to each other. However, axial movement of the components 28 and 30 with respect to each other (and the Z-axis) may still be possible.

In one embodiment, the inside wall of the barrel 34 includes two radial grooves 36 and 38, as depicted. In this embodiment, the core 32 includes radial ribs 40 and 42 on the outside wall of the core 32. Accordingly, the core 32 may be inserted into the interior of the barrel 34. When the core 32 is inserted into the barrel 34, the radial ribs 40 and 42 may engage the radial grooves 36 and 38, respectively. That is, the radial ribs 40 and 42 may be inserted into the radial grooves 36 and 38. The radial grooves 36, 38 and corresponding radial ribs 40, 42 aid in securing the attachment between the core 32 and the barrel 34 by preventing an axial movement of the core 32 with respect to the barrel 34 (and vice versa). Additionally, an interference fit between the core 32 and the barrel 34 may also aid in securing the core 32 to the barrel 34.

The grooves 36, 38 and ribs 40, 42 may also guide a radial rotation of the component 30 with respect to the core 32, or vice versa. That is, the grooves 36, 38 and ribs 40, 42 may allow the component 30 to more smoothly “turn” with respect to the core 32. In certain embodiments, the core 32 may include an angled portion 44. The angled portion 44 may aid in preventing the over-insertion of the core 35 into the barrel 34 by abutting (i.e., contacting) a bottom end of the barrel 34. Additionally, the angled portion 44 may limit the rotation of the flanges 20 and 22 by contacting, for example, the edges 46, 48 of the flanges 20, 22. The angled portion 44 may also include an opening 50 suitable for the insertion of an inflatable lumen. The inflatable lumen can be used, for example, to inflate one or more cuffs suitable for providing a seal between the cannula 14 and surrounding airway passages. By using on or more cuffs, substances may flow only through the cannula 14 (or other medical device), allowing better control over the type and amount of substances flowing into and out of the patient. Accordingly, all tracheal tube embodiments described herein may be manufactured to include features such as inflatable lumens or inflatable lines suitable for inflating one or more cuffs.

The core 32 may include an interior circular wall 52. The interior circular wall 52 in conjunction with an exterior wall 54, defines a channel 56. The channel 56 may be suitable for enabling the insertion of a circular wall disposed in the inside of rear of the end connector 18 into the channel 56, as described in more detail below with respect to FIG. 3. The component 14 (i.e., cannula) may include a proximal opening 58. The proximal opening 58 may be securely inserted into the rear of the core 32. Accordingly, the end connector 18, the barrel 34, and the core 32, and the proximal opening 58 of the cannula 14 may be placed approximately concentrically or co-axially with respect to each other, forming the tracheal tube 10.

FIG. 3 depicts a cross-section view of the components of the tracheal tube 10 described above with respect to FIGS. 1 and 2. As mentioned above, the cannula 14 may be axially inserted into the rear of the core 32. Accordingly, the core 32 may include a bottom circular channel 60 suitable for enabling the insertion of the cannula 14. The circular channel 60 may have an ID approximately equal to the OD of the walls of the proximal opening 58, thus enabling the insertion of the proximal opening 58 into the interior of the channel 60. In certain embodiments, a glue along with an interference fit between the outside walls of the circular channel 60 and the outside walls of the cannula 14 along may be used to securely attach the cannula 14 to the core 32. The core 32 and the attached cannula 14 may then be axially inserted into the rear of the component 30.

Once the core 32 has been inserted axially into the rear of the component 30, the radial ribs 40 and 42 of the core 32 may engage the grooves 36 and 38, respectively, of the second component 30. The insertion of the ribs 40 and 42 into the grooves 36 and 38 may prevent the detachment of the core 32 (and attached cannula 14) from the component 30. Additionally, an interference fit between the core 32 and the component 30 may aid in fastening the core 32 to the second component 30. The component 28 may then be inserted axially over the outer wall of the second component 30. As the component 28 axially “slides” over the outer wall of the second component 30, an interior circular wall 62 of the component 28 may be inserted into the circular channel 56 of the core 32. The teeth 35 may then engage a plurality of square notches 64 disposed positioned on the rear of the first component 28. The notches 64 are suitable for securing the teeth 34 in place so as to prevent radial rotation of the component 28 with respect to the component 30. However, the teeth 34 may be dislodged from the notches 64 by using an outwardly pulling axial force in the Z-axis. Indeed, a user may hold the flange 20 with one hand, and use the second hand to pull outwardly (i.e., along the Z-axis) on the flange 22. Such a pulling force may disengage the teeth 34 away from the notches 64. Once the teeth 34 have been disengaged, the flanges 20 and 22 may be rotated to desired angles.

Mechanical features such as a rib 66 and a notch 68 may prevent the complete detachment of the first component 28 from the second component 30 (and attached core 32 and cannula 14). The rib 66 is circumferentially disposed on the exterior of the circular wall 62, while the notch 68 is circumferentially disposed on the interior of the circular wall 54. As the first component 28 is moved away axially relative to the second component 30, the rib 66 may come into contact with the notch 68, preventing further motion. Once the user has rotated the flanges 20 and 22 to desired angles, the components 28 and 30 may then be re-attached. In the depicted embodiment, the user may re-attach the components 28 and 30 by using an axial pressing force along the Z-axis capable of re-engaging the teeth 34 to the notches 64.

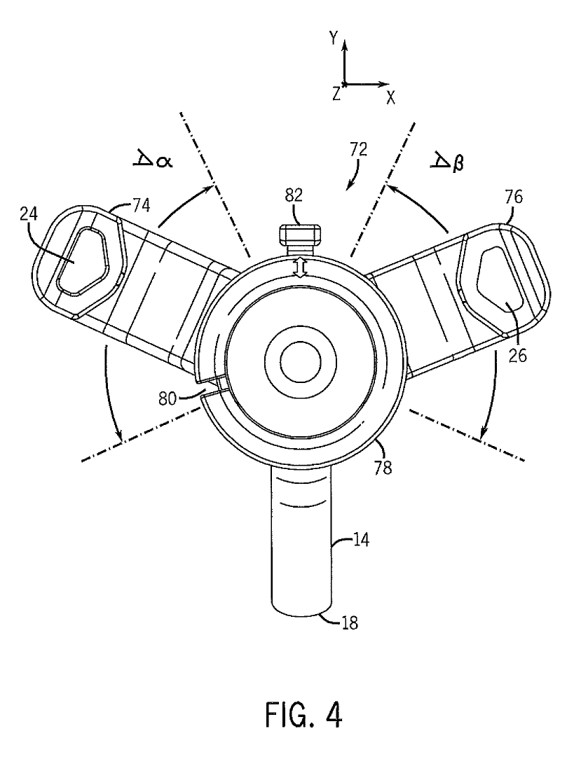

Mechanical features such as the rib 66 and the notch 68 may enable the user to quickly reposition the flanges 20, 22 because of the time savings associated with not having to completely detach and subsequently re-attach the first component 28 from the second component 30. Indeed, other mechanical features, such as those described in more detail with respect to FIG. 4, may allow a user to quickly change the angles of the flanges and then securely lock the flanges in place.

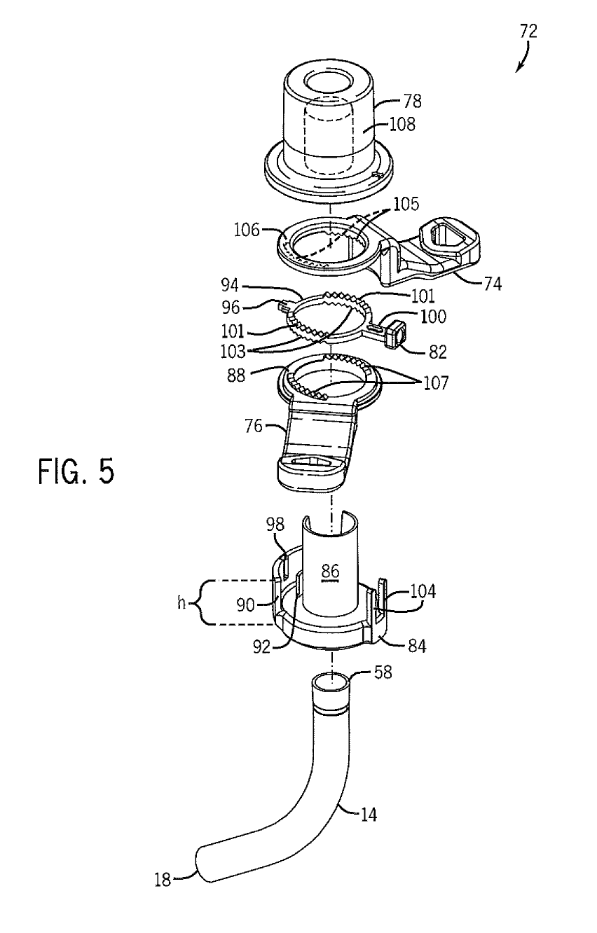

FIG. 4 depicts an embodiment of a tracheal tube 72 capable of quickly and securely repositioning a pair of angularly adjustable flanges 74, 76. In the depicted embodiment, the adjustable flanges 74 and 76 may be capable of rotating radially about an end connector 78 to any desired acute or obtuse angle, for example, angles α and β. The end connector 78 may include a 15 mm OD portion, i.e., male end connector that can couple with a standard 15 mm inner diameter (ID) connector, i.e., female end connector. It is to be noted that in other embodiments, the proximal end connector 78 may include a male connector portion of a different size, for example, 8 mm OD, 8.5 mm OD, and so on. Additionally, the end connector 78 may include a notch 80 suitable for enabling the insertion of a lumen, such as a lumen suitable for inflating a cuff, into the tracheal tube 72. The tracheal tube 72 also may include the cannula 14 having the distal opening 18. Accordingly, the cannula 14 may be inserted into a patient's trachea, forming an artificial airway. In certain embodiments, the tracheal tube 72 may include a locking and unlocking feature, such as a flexible ring 82. Indeed, the flexible ring 82 may enable a fast and secure locking and unlocking of the adjustable flanges 74 and 76, as described in more detail below with respect to FIG. 5.

FIG. 5 is an exploded view of the various components of the tracheal tube 72 of FIG. 4. More specifically, the figure depicts from top to bottom components 78, 74, 82, 76, 84 and 14. The tracheal tube 72 may be assembled by positioning the flange 76 co-axially or concentrically on top of the base plate 84 (i.e., contacting the base plate 84) such that a crescent-shaped or “C” shape wall 86 of the base plate 84 is inserted through a center opening of an annular disk 88 of the flange 76. Such positioning enables the annular disk 88 to be disposed inside a semi-circular channel formed by an outside surface of a crescent-shaped wall 90 and an inside surface of a crescent-shaped wall 92 of the base plate 84. The flexible ring 82 may then be co-axially or concentrically disposed on top of the flange 76 (i.e., contacting the flange 76), such that the crescent-shaped wall 86 of the base plate 84 is inserted through a center opening of an annular portion 94 of the flexible ring 82. Likewise, the annular portion 94 of the flexible ring 82 may also be disposed in the semi-circular channel formed by the outside surface of the crescent-shaped wall 90 and the inside surface of the crescent-shaped wall 92 of the base plate 84. Further, a rectangular tab 96 projecting radially from the annular portion 94 may be disposed inside a notch 98 of the crescent-shaped wall 90. A rectangular tab 100 projecting radially from the flexible ring 82 may be positioned in between two posts 104 of the base plate 84. The flange 74 may then be co-axially or concentrically disposed on top of the flexible ring 82 (i.e., contacting the flexible ring 82) such that the crescent-shaped shape wall 86 of the base plate 84 is inserted through a center opening of an annular disk 106 of the flange 74. The annular disk 106 of the flange 74 may also be placed inside of the semi-circular channel formed by the outside surface of the crescent-shaped wall 90 and the inside surface of the crescent-shaped wall 92 of the base plate 84. Accordingly the wall 90 may include a height h approximately equal to the height of the components 74, 82, and 76 when positioned to be on top of one another (i.e., “stacked” on top of each other). Thus, the top-most component, i.e., flange 74, may not extend past the top of the wall 90.

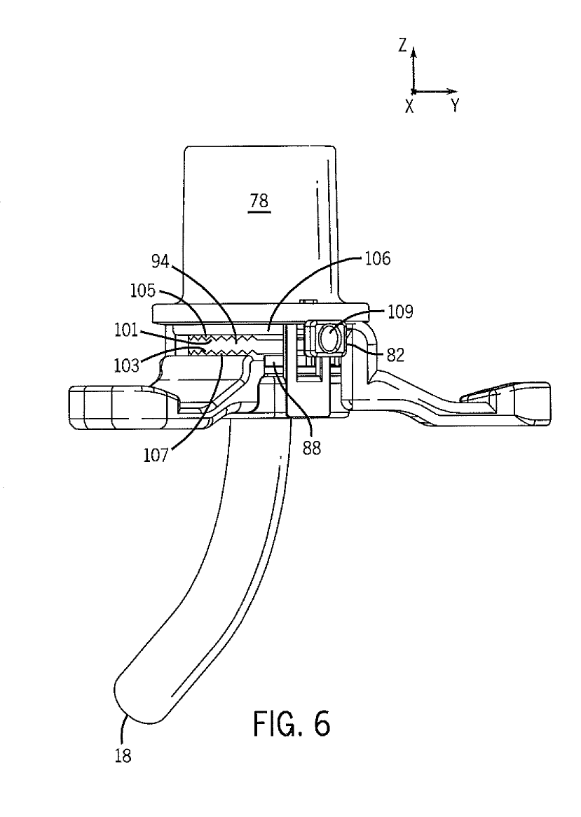

The end connector 78 may then be co-axially or concentrically connected to the proximal end of the cannula 14. In this embodiment, the proximal opening 58 of the cannula 14 may be inserted into the rear end of the end connector 78. Consequently, the distal end of the cannula 14 (with the end connector 78 attached), may be inserted through the interior of the crescent-shaped wall 86 of the base plate 84 so as to emerge out of the distal end of the base plate 84. The end connector 78 may then be co-axially or concentrically disposed on top of the flange 74 such that the crescent-shaped wall 86 of the base plate 84 is inserted into the interior of a channel 108 of the end connector 78. That is, the base plate 84 along with the stacked components 74, 82, and 76 may be inserted into the rear of the end connector 78. In certain embodiments, the end connector 78 may then be glued to the base plate 84. In these embodiments, glue may be used so as to securely fasten the crescent-shaped wall 86 into the channel 108. When the tracheal tube 72 is thus assembled or manufactured, the flexible ring 82 may be used to lock and unlock the flanges 74 and 76, as described in more detail below with respect to FIG. 6. It is to be understood that any order of assembly may be used that allows the components of the tracheal tube 72 to be disposed as described with respect to FIG. 5.

FIG. 6 depicts a perspective view of the assembled tracheal tube 72. In the depicted embodiment, the flexible ring 82 is shown disposed between the annular disk 88 of the flange 76 and the annular disk 106 of the flange 74. The flexible ring 82 includes an upper set of protrusions (i.e., triangular teeth) 101 and a lower set of protrusions 103 (i.e., triangular teeth). The teeth 101 are positioned so as to engage a similar set of teeth 105 included in the upper flange 74. Likewise, the teeth 103 are positioned to engage a set of teeth 107 included in the lower flange 76. It is to be understood that, in other embodiments, the protrusions 101, 103, 105, and 107 may have other shapes such as square shapes, curved shapes, and so forth. Indeed, any shapes capable of interlocking with each other may be used.

The flexible ring 82 may also include a button 109. The button 109 may enable a user to quickly and securely disengage and subsequently reengage the teeth 101, 103, 105, and 107 so as to lock and unlock the flanges 74 and 76. In order to unlock the flanges 74 and 76, the button 109 may be pressed inwardly towards the center of the end connector 78. The pressing force may distort the shape of the flexible ring 82, causing the teeth 101 and 103 to disengage from the teeth 105 and 107, respectively. Indeed, the pressing force may be capable of, for example, causing sections of the annular portion 94 of the flexible ring 82 to press outwardly against the annular disk 88 and/or the annular disk 106. The outward pressure may move the flange 74 and/or the flange 76 in an opposite axial direction relative to each other along the Z-axis, thus unlocking the flanges 74 and 76. The user may then rotate the flanges 74 and 76 to desired angles. Once the flanges are placed at desired angles, the button 109 may be released. The lack of the pressing force on the flexible ring 82 allows the annular ring 82 to return to its resting state, thus enabling the teeth 101 and 103 in re-engaging the teeth 105 and 107, respectively. Accordingly, the release of the button 109 may lock the flanges 74, and/or 76 in place.

In certain embodiments, the teeth 101 and 103 may be disposed throughout the entire circumference of the annular disk 94 of the ring 82. In these embodiments, it may be possible to rotate and lock each of the flanges 74 and/or 76 360° about the Z-axis. Such enhanced flange positioning capabilities may enable a user to comformably fit the tracheal tube flanges to any radial orientation, thus allowing for increased comfort and a more secure placement on the patient's neck.

As mentioned above, the components of the tracheal tube 72, such as components 78, 74, 82, 76, 84 and 14 may be molded, cast, milled, and so forth. Some example materials that may be used in the manufacturing of the tracheal tube 72 components include ABS, PVC, PET, LDPE, polypropylene, silicone, neoprene, and polyisoprene. In certain embodiments, the flexible ring 82 may be of a lesser shore hardness than other components such as the base plate 84 and flanges 74 and 76, thus allowing for increased flexibility of the ring 82 as compared to the other components 84, 74, and 76. The increased flexibility allows the ring 82 to deform with less force, thus enabling a quicker unlocking and locking of the adjustable flanges 74 and 76.

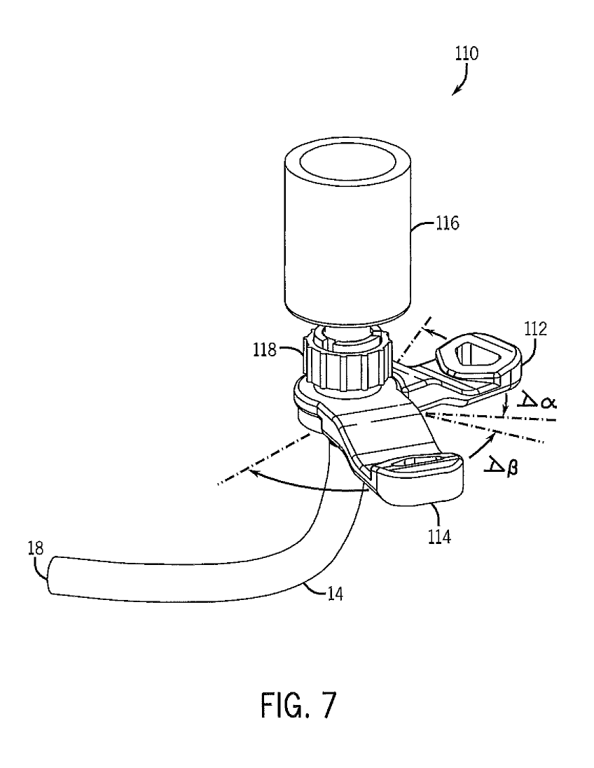

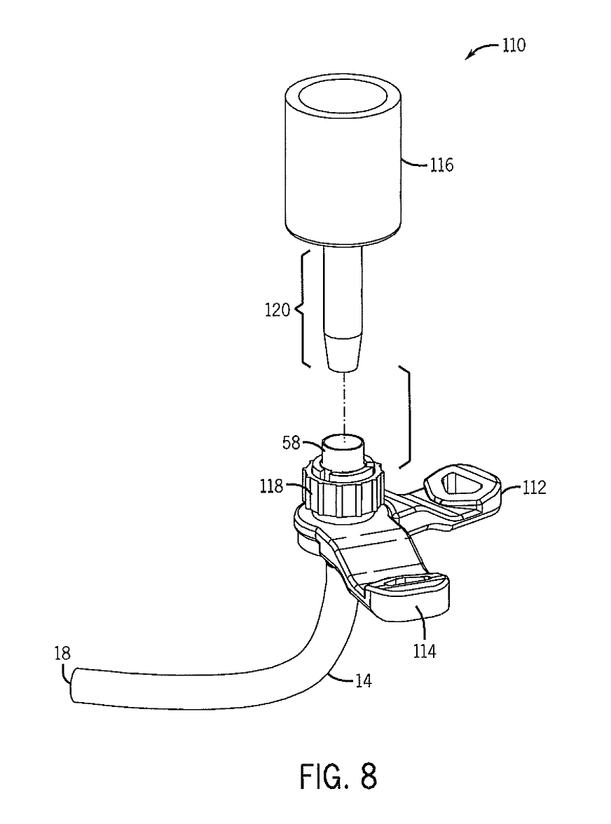

FIG. 7 is a perspective view of an embodiment of a tracheal tube 110 having angularly adjustable flanges 112, 114. In the depicted embodiment, the flanges 112 and 114 may be radially rotated about an end connector 116. Indeed, the flanges 112 and 114 may be rotated 360° about the end connector 116. Further, the end connector 116 may be an extendable end connector 116. That is, the end connector may extend axially from the proximal end of the tracheal tube 110, thus increasing the length of the proximal end of the tracheal tube 110. The repositionable tracheal tube extension 116 provides a clinician with an increased working space in the area in front of the tracheal tube, thus allowing for rapid attachment of devices such as ventilators, manual respirators, suctioning equipment, nebulators, vaporizers, tee connectors, and so forth, to the proximal end of the tracheal tube. Further, the repositionable tracheal tube extension 116 may decrease patient discomfort during regular use by extending the distance between attachments to the tracheal tube and the frontal neck region. The increased distance provides for additional freedom of movement of the head and minimizes physical contact between the chin, stoma, and/or neck with the attachments at the proximal end of the tracheal tube. The repositionable tracheal tube end connector 116 may be extended, retracted, and/or replaced, for example, by utilizing a nut 118 as described in more detail below with respect to FIG. 8. Additionally, the nut 118 allows for the adjustable flange 112 and the adjustable flange 114 to be radially repositioned to any angle α and/or β, respectively.

FIG. 8 is a perspective view of the tracheal tube 110 with the end connector 116 removed. Indeed, the end connector 116 may be removed and replaced, for example, with an end connector having a different size (e.g., 8 mm, 8.5 mm, 15 mm) or receptacle type (e.g., male end connector, female end connector). Further, the end connector 116 may extend or retract to a desired proximal length. Accordingly, the end connector 116 may include a bore 120 capable of connecting to the cannula 14. It is to be noted that bore 120 may be manufactured in a variety of lengths suitable for applications desiring longer extensions or shorter extensions. Indeed, the bore 120 may be manufactured having any suitable length. The bore 120 may be inserted axially into the proximal opening 58 of the cannula 14 and positioned at a desired length. The nut 118 may then be rotated, causing a radially compressive force to tightly secure the bore 120 to the cannula 14. Such a compressive force enables the “locking” of the bore 120 to the cannula 14. In certain embodiments, a clockwise rotation of the nut 118 may be result in the compressing force securing the bore 120 to the cannula 14. In these embodiments, a counterclockwise rotation of the nut 118 may be capable of releasing (i.e., “unlocking”) the bore 120 from the cannula 14.

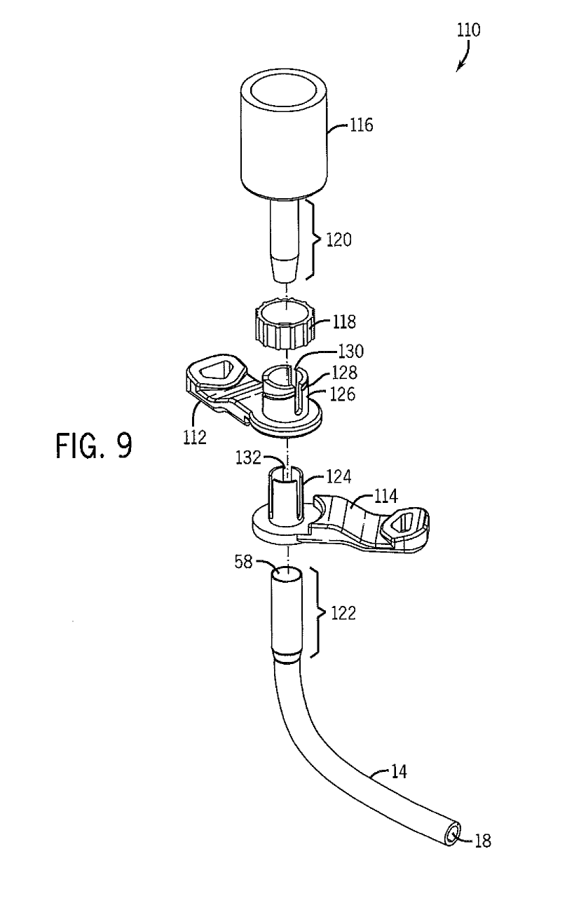

In other embodiments, a counterclockwise rotation of the nut 118 may lock the bore 120 to the cannula 14, while a clockwise rotation of the nut 118 may unlock the bore 120 from the cannula 14. By “turning” the nut to lock and unlock the bore 120 form the cannula 14, the user may quickly reposition or remove the end connector 116. Additionally, the nut 118 may also enable the repositioning of the flanges 112 and/or 114, as described in more detail below with respect to FIG. 9.

FIG. 9 is an exploded view of embodiments of the components of the tracheal tube 110. More specifically, the figure depicts from top to bottom components 116, 118, 112, 114, and 14. In the depicted embodiment, the tracheal tube 110 may be assembled by inserting the proximal opening 58 of the cannula 14 concentrically or co-axially through the flange 114. More specifically a proximal portion 122 of the cannula 14 may be axially inserted through a circular wall 124 of the flange 114. Accordingly, the OD of the proximal portion 122 may be smaller or approximately equal to the ID of the circular wall 124. In one embodiment, the proximal portion 122 may have a larger OD than the OD of the remainder portions of the cannula 14. In this embodiment, the ID of the portion 122 may be approximately the same as the ID of the remainder of the cannula 14. Accordingly, the walls of the portion 122 may be thicker than the remainder walls of the cannula 14. The thicker walls of the portion 122 may enable the portion 122 to be repeatedly compressed without exhibiting cracking or braking. In another embodiment, the OD of the portion 122 may be approximately the same as the OD of the remainder portions of the cannula 14. In such an embodiment, the distal end and the proximal end of the tracheal tube 110 may be interchangeable. That is, the opening 58 may be used as the distal end and the opening 18 may be used as the proximal end, or vice versa. In yet another embodiment, the portion 122 may have a smaller OD than the remainder of the cannula 14, for example, to minimize airway dead space. Minimizing airway dead space allows for less energy expenditure and may result in a decreased work-of-breathing (WOB).

The flange 112 may then be concentrically or co-axially disposed on top of the flange 114. In this example, the portion 122 and the circular wall 124 may be inserted through a circular wall 126 of the flange 112. Accordingly, the OD of the circular wall 124 may be smaller or approximately equal to the ID of the circular wall 126. The nut 118 may then be concentrically or co-axially positioned on top of the circular wall 126 of the flange 112. The bore 120 of the end connector 116 may then be axially inserted into the proximal opening 58 of the cannula 14. In the depicted embodiment, the portion 122 of the cannula 14 may have a length greater than the height of the circular walls 124, 126 and the nut 118 and may be capable of protruding through the top of the circular walls 124, 126 and the nut 118. By protruding out of the circular walls 124, 126 and the nut 118, the portion 122 may enable a faster and simple insertion of the bore 120 into the proximal opening 58.

Once the bore 120 is axially inserted at a desired depth into the proximal opening 58, the flanges 112 and 114 may be positioned at desired angles α and β to more comformably fit the patient's neck anatomy. The flanges 112, 114 and the bore 120 may then be secured in place by rotating the nut 118. The nut 118 may include a helical ridge in the interior of the nut 118. The helical ridge may engage a helical groove (e.g., thread) 128 inscribed in the exterior circular wall 126 of the flange 112. The groove 128 may decrease in depth as the groove 128 follows the contour of the circular wall 126 from the top of the circular wall 126 to the base of the circular wall 126. That is, the groove 128 near the top of the circular wall 126 may be of a greater depth than the groove 128 near the base of the circular wall 126. Accordingly, the deeper groove 128 near the top of the circular wall 126 may prevent the removal of the nut 118.

As the nut 118 is rotated (i.e., “turned”) so as to move axially closer the base of the circular wall 126, an increased compression fit between the cannula 14, the flanges 112, 114, and the bore 120 may result. Further, the flange 112 may include slots 130 while the flange 114 may include slots 132 that enable the circular walls 126 and 124, respectively, to move inwardly (i.e., towards the center of the proximal opening 58). Accordingly, the slots 130 and 132 may enable an enhanced compression fit by allowing for a higher compression force that may be used to secure the cannula 14, the flanges 112, 114, and the bore 120. Thus, by using a nut 118, a user may quickly unscrew the nut 118, position the flanges 112, 114, and the end connector 116 to desired placements, and then screw the nut 118 to “lock in” the desired placements. Such capability may allow the user to easily readjust the flanges 112, 114, and end connector 116 so as to avoid neck obstructions and to better fit any number of neck anatomies.

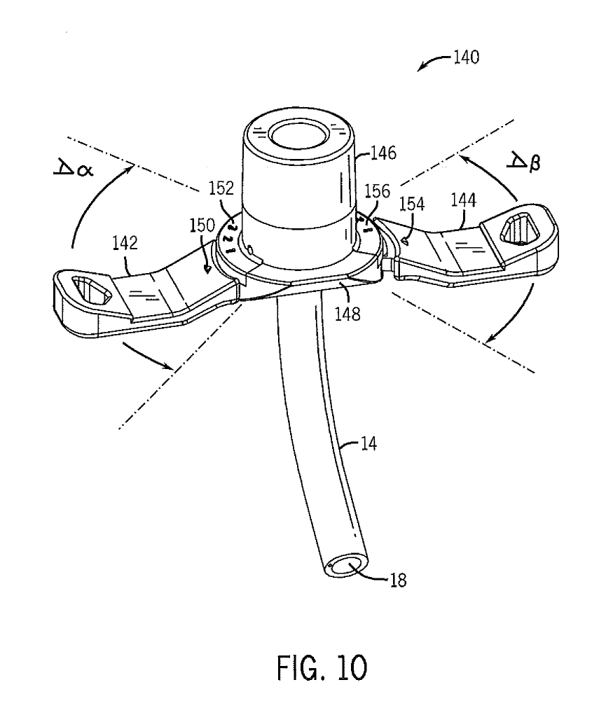

FIG. 10 is a perspective view of another embodiment of a tracheal tube 140 having angularly adjustable flanges 142 and 144. In the depicted embodiment, the tracheal tube 140 includes an end connector 146 disposed on a base plate 148. Additionally, the flange 142 may include an angle indicator 150. The angle indicator 150 may enable the user to more precisely position the flange 142 by lining up the angle indicator with an angle marking 152. Likewise, the flange 144 may include an angle indicator 154, which may aid the user to more precisely position the flange 144 by lining up the angle indicator with an angle marking 156. Indeed, a number of discrete angles, such as angles α and β may be marked by the angle markings 152 and 156, respectively. The use of angle markings, such as angle markings 152 and 156, may enable the re-positioning of the flanges 142 and 144 to various discrete angular positions.

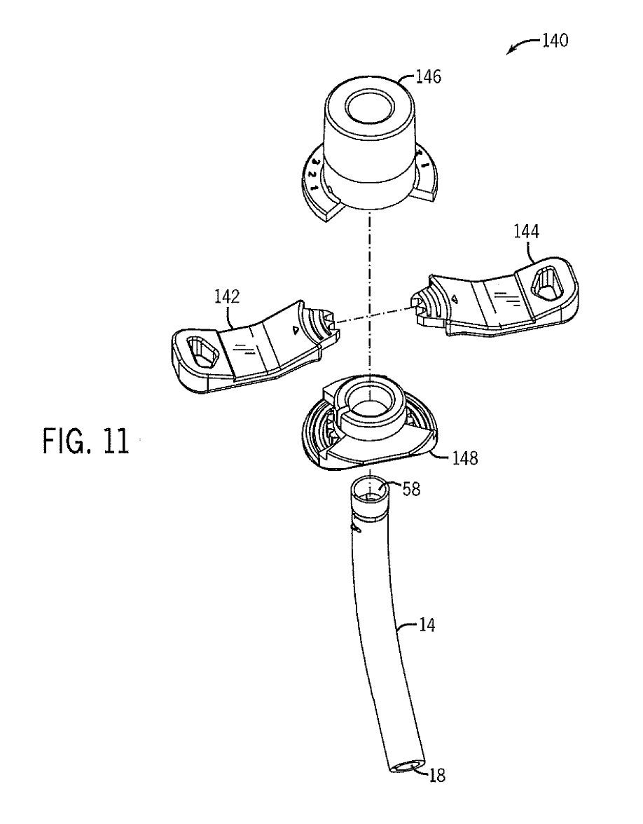

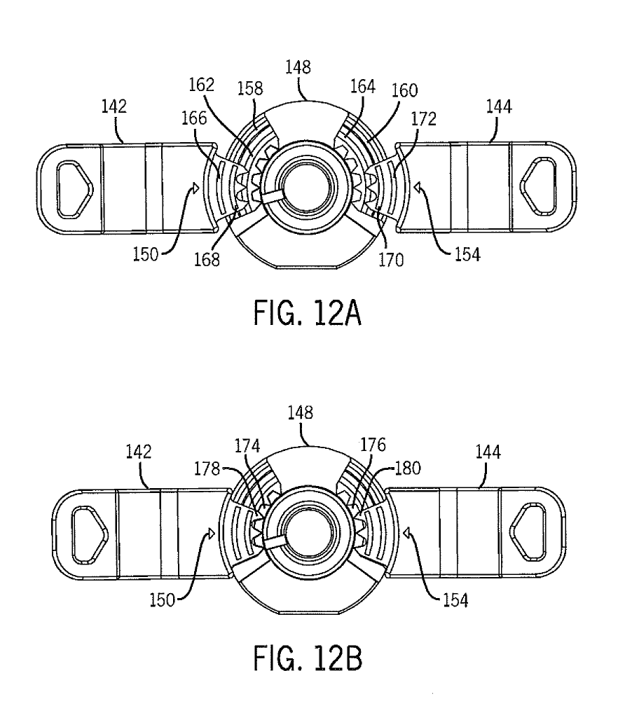

FIG. 11 is an exploded view of embodiments of components of the tracheal tube 140. More specifically, the figures shows, from top to bottom, components 146, 142, 144, 148, and 14. The figure is also illustrative of how the various components that may be assembled to form the tracheal tube 140. The tracheal tube 140 may be assembled by first inserting the proximal opening 58 of the cannula 14 into the rear of the base plate 148. The cannula 14 may then be glued to the rear of the base plate 148. The two flanges 142 and 144 may then be positioned on top of the base plate 148, one at each side of the base plate 148. The end connector 146 may then be glued to the base plate 148 (and to the cannula 14). The flanges 142 and 144 are left unglued so that they may be re-positioned and locked in place as described below with respect to FIGS. 12A and 12B.

FIG. 12A is a frontal view of the flanges 142, 144 and the base plate 148. In certain embodiments, such as those depicted in FIG. 12A, the tracheal tube 140 may have been manufactured to have the flanges 142 and 144 already pre-inserted into a first groove 158 and 160, respectively. That is, the flange 142 may have been pre-inserted into the first groove 158 on one side of the base plate 148, and the flange 144 may have been pre-inserted into the first groove 160 on the opposite side of the base plate 148. When a flange, such as the flange 142 or 144, is disposed on the first groove, such as the groove 158 or 160, the flange may still be moved radially, following the groove 158 or 160. Accordingly, the user may move the flanges 142 and/or 144 to a desired angle by using angle indicators 150 and/or 154. Once the desired angle is reached, the user may then exert a radial force by holding the flanges 142 or 144 and pressing inwardly towards the center of the base plate 148. Such a force may move the flanges 142 or 144 closer to the center of the base plate 148 and may then cause the flanges 142 or 144 to engage a second groove (e.g., groove 162 or groove 164), as depicted in FIG. 12B.

In some embodiments, the flanges 142 and 144 may include radial ridges on the top surface and on the bottom surface of the flange ends, such as the top surface ridges 166, 168, 170, and 172. The ridges are capable of engaging the grooves. That is, the ridges are capable of being inserted into the grooves. Accordingly, the top surface ridges 166, 168, 170, and 172 may engage grooves on the bottom end (i.e. rear) of the end connector 146, while the bottom ridges (not shown) may engage the grooves 158, 160, 162, and 164 of the base plate 148.

FIG. 12B is a frontal view illustrating the flanges 142 and 144 disposed on the base plate 148 and engaging teeth 174 and 176 of the base plate 148, respectively. In the depicted embodiment, the user may have exerted a radial force inwardly towards the center of the base plate 148, thus moving the flanges 142 into the second groove 174 and the flange 144 into the second grooves 176. That is, the flange 142 may now be engaging both grooves 158 and 162, while the flange 144 may now be engaging both grooves 160 and 164. Accordingly, teeth 178 of the flange 142 may engage the teeth 174 of the base plate 148. Likewise, the teeth 180 of the flange 144 may engage the teeth 176 of the base plate 148. The engagement of the flange teeth with the base plate teeth securely locks the flanges 142 and/or 144 into desired positions on the base plate 148, thus preventing movement of the flanges 142 and 144 about the base plate 148.

In one embodiment, the flanges 142 and 144 in combination with the grooves 158, 160, 162, and 164 of the base plate 148 (and the grooves of the end connector 146) may enable “one-way” insertion of the flanges 142, 144. That is, once the flange 142 has been fully inserted into the base plate 148 so that the flange teeth 178 engage the base plate teeth 174, the flange 142 may become permanently affixed to the base plate 148. Similarly, once the flange 144 has been fully inserted into the base plate 148 so that the flange teeth 180 engage the base plate teeth 176, the flange 144 may become permanently affixed to the base plate 148. In this embodiment, the second grooves 162 and 164 may be deeper grooves than the first grooves 158 and 160. The bottom ridges and the top ridges 166, 168, 170, and 172 may also include features, such as a barb, that may further prevent the removal of the flanges 142, and 144 from the base plate 148 and the end connector 146. Such a technique for permanently affixing the flanges 142 and/or 144 to the base plate 148 and the end connector 146 may prevent, for example, pediatric patients from inadvertently removing the flanges 142 and/or 144.

In another embodiment, the flanges 142, 144 in combination with the grooves 158, 160, 162, and 164 of the base plate 148 (and the grooves of the end connector 146) may enable repositioning of the flanges 142, 144. In this embodiment, an outwardly pulling force may be used to remove the flanges 142 and/or 144 from the base plate 148 and the end connector 146. Angle indicators, such as the angle indicators 150 and 154, may then be used to aid in aligning the flanges into a desired angular position. The flanges 142 and/or 144 may then be positioned into the desired angle by exerting an inwardly pressing force, as described above, suitable for engaging the flange teeth with the base plate 148 teeth. Such repositioning capabilities allow the tracheal tube 140 to be adjusted, for example, so as to avoid certain neck obstructions such as anatomical obstructions. Further, the tracheal tube 148 may be used to fit a variety of patient types, including pediatric and adult patients.

In yet other embodiments, the flanges 142, 144 and bottom plate 148 may not include any teeth but may use techniques, such as an interference fit, that allow for a more passive movement of the flanges. That is, the flanges 142 and 144 may move but the movement may be more restrained due to the interference fit. In these embodiments, an interference fit between the grooves on the end connector 146 and the grooves on the base plate 148 (e.g., grooves 158, 160, 162, and 164) with the top and bottom ridges (e.g., ridges 166, 168, 170, and 172) may allow a more restrained movement. Additionally, the interference fit may become stronger at certain angles, thus further restraining the movement to certain more optimal positions. Indeed, all of the tracheal tube embodiments described herein, such as in FIGS. 1-19, may include similar fastening techniques suitable for allowing passive movements of the flanges and/or optimal angle positioning features.

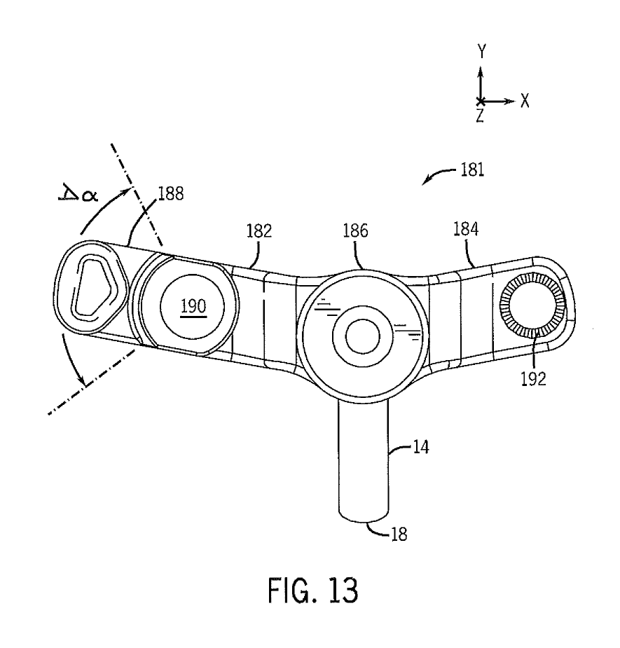

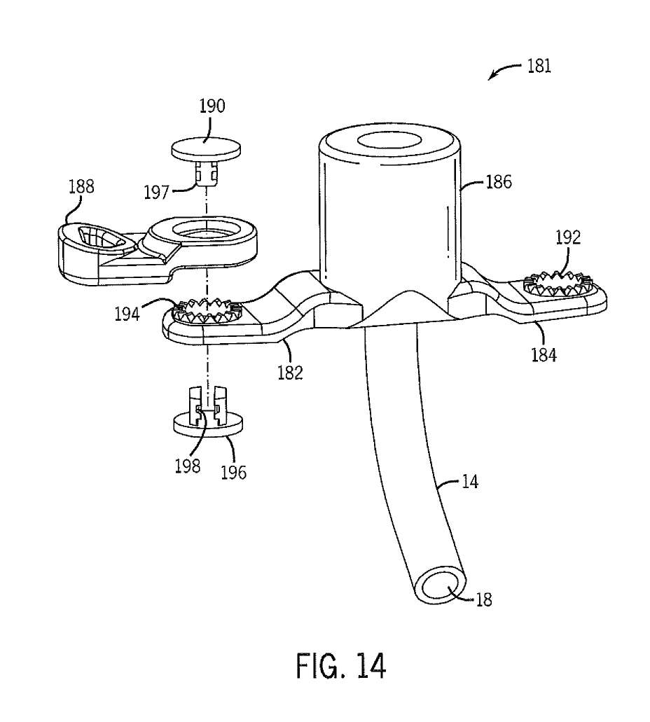

FIG. 13 is a frontal view of an embodiment of a tracheal tube 181 having flanges 182 and 184 positioned to extend radially from an end connector 186 and capable of including angularly adjustable flange extensions, such as flange extension 188. A fastener, such as an upper button 190, may be used in securing the flange extension 188 to the flange 182. An equivalent flange extension and button may be disposed on flange 184. Additionally, a set of teeth 192, enabling the secure attachment of the flange extension, may be disposed circumferentially around an opening in the flange. The teeth 192 may engage a similar set of teeth disposed on the bottom surface of the flange extension 188, thus limiting or preventing the rotation of the flange extension when the flange extension is locked in place, as described in more detail below with respect to FIG. 14. Indeed, features such as the teeth 192 and the button 190 may enable the secure positioning of the flange extensions at a variety of angles relative to the X-Y axis, such as angle α. Indeed, in certain embodiments, the flange extension 188 may be positioned at any angle α between approximately 0° and approximately 360°. Further, the angle extension 188 may be used with any adjustable flange embodiments, such as the embodiments described in FIGS. 1-12B above. That is, the adjustable flanges described in FIGS. 1-12B may further include the flange extension techniques discussed herein, such as the flange extension 188, teeth 192, and buttons 190.

FIG. 14 is an exploded view of the tracheal tube 181 of FIG. 13. As mentioned above, the tracheal tube 181 may include a pair of flange extensions, such as the flange extension 188. The user may wish to position the flange extension 188 at a specific angle, such as angle α. Accordingly, the user may position the flange extension at the desired angle and dispose the flange extension 188 co-axially or concentrically on top of teeth 194. The upper button 190 may then be fastened to or “clipped” to a lower button 196 so as to securely lock the flange extension 188 in place. The upper button 190 may include a set of protrusions 197 suitable for engaging a set of grooves 198 in the lower button 196. Accordingly an axial pressing force directed towards the top of the button 190 and/or 196 may be used to fasten the flange extension 188. For example, a user may press the two buttons 190 and 196 between a thumb and an index finger to cause the protrusions 197 to engage the grooves 198. The flange extension 188 may then be securely locked to the flange 182. Indeed, a user may use one hand to hold the flange extension 188 at a desired position and may then use the other hand to press the two buttons 190 and 196 together so as to fasten the flange extension 188 to the flange 182.

In one embodiment, the locking of the button 190 to the button 196 may be “one-way”. That is, once the buttons 190 and 196 are locked together, it may be difficult to unlock the buttons 190 and 196 without resorting to special tools. In this embodiment, pediatric and/or psychiatric patients may thus be prevented from removing or repositioning the flange extensions. In other embodiments, the flange extension 188 may be repositioned by disengaging the buttons 190 and 196. In these embodiments, the protrusions 197 of the top button 190 may be smaller protrusions or may not extend as deeply into the grooves 198 of the bottom button 196. Additionally, the grooves 198 may be of lesser depth. Further, the buttons 190 and/or 196 may include other manually grippable features such as handles, ridges, slots, and so forth, suitable for disengaging the button 190 from the button 196. The button 190 may then be disengaged from the button 196 by applying an axial pulling force capable of separating the two buttons 190, 196. Such features may allow the flange extension 188 to be repositioned to a new angle, as desired.

In other embodiments, the flanges 182, 184 may not include the teeth 192 or the teeth 194. In these embodiments, the connection between the buttons 190 and 196 may result in a button fastening force of a strength suitable for maintaining a desired angle. That is, the button fastening force may be of a strength suitable for resisting certain radial forces. In these embodiments, the user may still radially reposition the flange extension 188 by exerting a radial force sufficient to overcome the button fastening force. Such capabilities may allow for a faster repositioning of the tracheal extension 188.

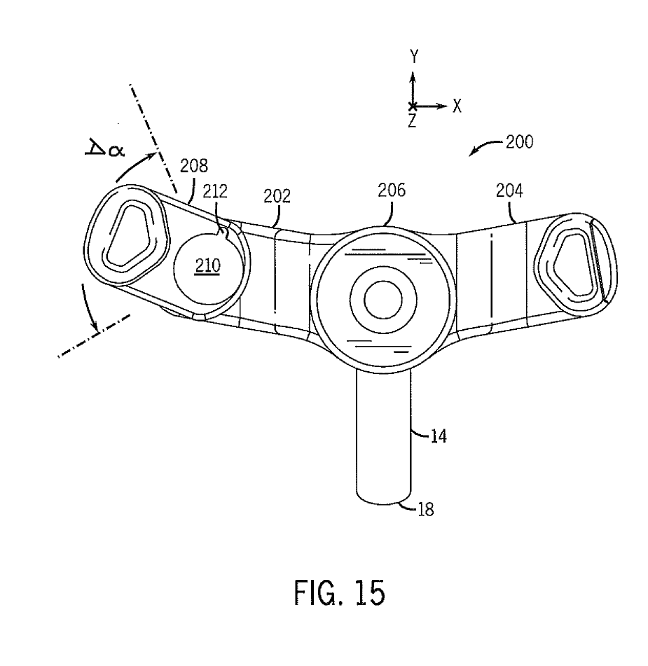

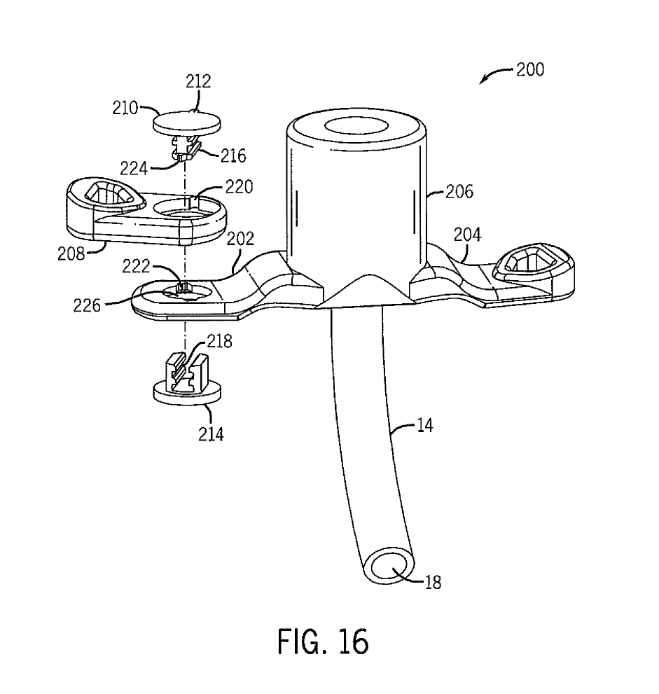

It is to be understood that the flange extension 188 may be manufactured in different lengths. Longer extensions may be used, for example, in neck anatomies having wider circumferences. Shorter extensions may be used, for example, in pediatric patients. Additionally, two flange extensions 188 may be used. One flange extension may be connected to the flange 182 and the second flange extension may be connected to the flange 184. In other embodiments, such as those depicted in FIGS. 15 and 16, a single flange extension may be used.

FIG. 15 illustrates an embodiment of a tracheal tube 200 having flanges 202 and 204 positioned radially about an end connector 206. In this embodiment, the tracheal tube 200 includes a single angularly adjustable flange extension 208. That is, the flange 204 is a fixed flange while the flange 202 may include the adjustable flange extension 208. Having one adjustable flange and one fixed flange 204 may limit the number of interstices (e.g., spaces or gaps) included in the tracheal tube 200, thus reducing the locations that may harbor bacteria. In the depicted embodiment, the flange extension 208 may be fastened in place at any angle α on the X-Y plane using techniques such as a top button 210 having a notch guide 212. The notch guide 212 may further aid in securing the flange extension 208, as described in more detail below with respect to FIG. 16.

FIG. 16 is an exploded view of the tracheal tube 200 depicting an embodiment of the flange extension 208. In the depicted embodiment, the flange extension 208 may be fastened to the flange 202 through the use of the top button 210 having the notch guide 212 and a bottom button 214. The top button 210 may include protrusions such as protrusions 216 suitable for engaging grooves, such as grooves 218 of the bottom button 214. Accordingly, the user may position the flange extension 208 on top of the flange 202 at a desired angle. The user may then fasten the flange extension 208 to the flange 202 by directing an axial pressing force towards the top surfaces of the buttons 210 and 214. For example, a user may press the two buttons 210 and 214 between a thumb and an index finger to cause the protrusions 216 to engage the grooves 218.

In the depicted embodiment, the flange extension 208 includes a notch 220 suitable for mating with the notch guide 212 of the button 210. As the button 210 is pressed axially through the flange 202 and into the grooves 218 of the button 214, a protrusion 224 of the button 210 may engage the teeth 222 of the flange 202. At approximately the same time, the notch guide 212 may engage the notch 220 of the flange extension 208. The engagement of the protrusion 224 to the teeth 222 may prevent a radial movement of the button 210 with respect to the flange 202. Likewise, the engagement of the notch guide 212 to the notch 220 may prevent a radial movement of the flange extension 208 with respect to the button 210. Finally, the engagement of the protrusions 216 to the grooves 218 may prevent an axial movement of the buttons 210 and 214 with respect to the flange 202 (and the flange extension 208). Accordingly, the notch 220, notch guide 212, and teeth 222 may assist in maintaining the flange extension 208 at a desired angle α.

In one embodiment, the teeth 222 may be disposed 360° circumferentially about an opening 226, thus allowing the flange extension to be positioned at a number of desired angles α. In another embodiment, such as the depicted embodiment, the teeth 222 may be disposed at desired positions around the opening 226, thus limiting the flange extension 208 to certain discrete angles. Such an embodiment may enable the user to more quickly position the flange extension 208 at an angle α.

In certain embodiments, the buttons 210 and 214 may enable a “one way” locking of the flange extension 208 at a desired angle α. In these embodiments, once the button 210 has been inserted through the flange extension 208 and the flange 202 so as to fully engage with the button 214, the buttons 210 and 214 may then be locked in place. For example, the grooves 218 may be barbed grooves 218 that allow for the entry of the protrusions 216 in one direction but that prevent the protrusions 216 from moving in the opposite direction. In other embodiments, the buttons 210 and 214 may be disengaged or unlocked from each other. For example, the buttons 210 and/or 214 may include grippable features enabling the exertion of a pulling force capable of disengaging the buttons 210 and/or 214 from each other.

In other embodiments, the teeth 222 may not be used. In these embodiments, a certain amount of movement of the flange extension 208 with respect to the flange 202 may be allowed. The movement may be restrained to certain angles or angle ranges by the use of the notch guide 212 and the notch 220. That is, the flange extension may move but the movement may be constrained as the notch guide 212 encounters the sides of the notch 220. Further, a button fastening force created by the connection between the button 210 and the button 214 may have a strength suitable for aiding in maintaining the desired angle or angle range.

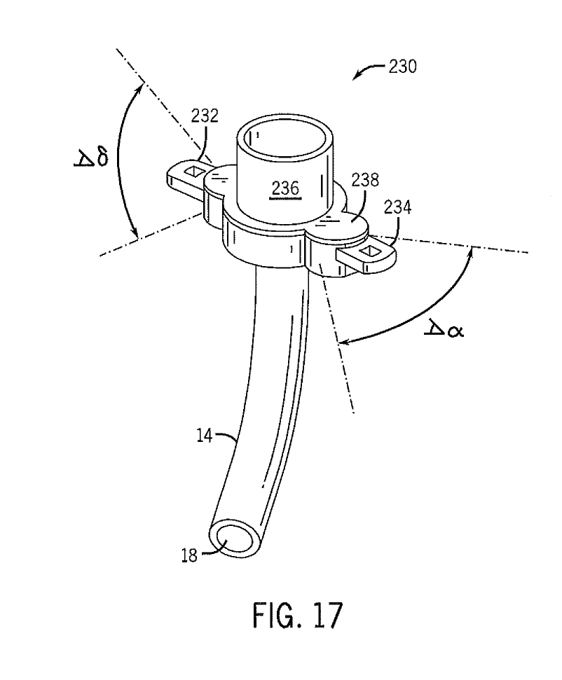

FIG. 17 depicts an embodiment of a tracheal tube 230 having enclosed angularly adjustable flanges 232 and 234 and an end connector 236. The adjustable flanges 232 and 234 may be enclosed with a cover 238 so as to aid in preventing bacteria and/or fluids from entering into interstices of the adjustable flanges 232 and 234. In this embodiment, the adjustable flanges 232 and 234 may move axially (defining an angle δ) as well as radially (defining an angle α) with respect to the end connector 236. That is, the angle δ defines an up or down movement (i.e., axial movement) of the adjustable flanges 232 and 234, while the angle α defines a left or right movement (i.e., radial movement) of the adjustable flanges 232 and 234. Such capabilities allow for two degrees of freedom in the adjustability of the flanges 232 and 234. Accordingly, the depicted embodiment is capable of an enhanced patient comfort and placement of the tracheal tube 230 by enabling an axial movement in addition to the radial movement of the flanges 232 and 234. Such capabilities allow the patient additional axial freedom of movements (e.g., two degrees of freedom) of the tracheal tube 230 with respect to the neck, increasing patient comfort. It is to be understood that, in other embodiments, more degrees of freedom may be used to adjust the flanges 232 and 234, such as three, four, five, or six degrees of freedom.

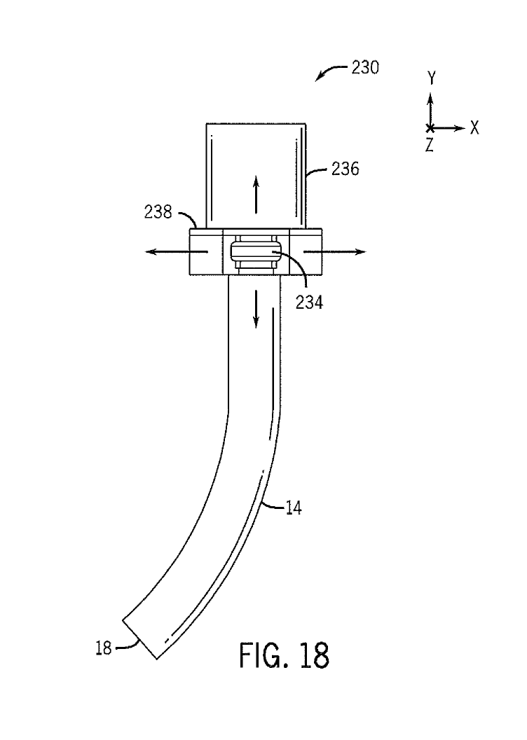

FIG. 18 is a side view of an embodiment of the tracheal tube 230. As depicted the flanges, such as the flange 234, is capable of moving along the X-axis (i.e., moving radially with respect to the end connector 236) as well as along the Y-axis (i.e., moving axially with respect to the end connector 236). The radial movement allows the flange 234 to be positioned at a desired angle α, for example, to more comformably fit the tracheal tube 230 to different neck anatomies. The axial movement allows the flange 234 to be positioned at a desired angle δ, for example, to more comformably fit over a protrusion such as a catheter strap. Additionally, the patient's neck movement may be made more comfortable because the adjustable flanges 232 and 234 allow for an increase in the movement of the neck axially with respect to the end connector 236, while still maintaining a secure attachment of the tracheal tube 230 to the neck.

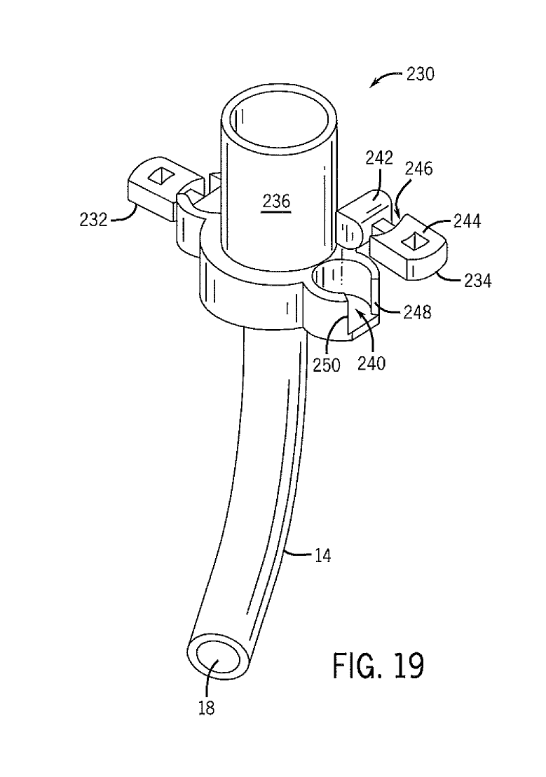

FIG. 19 is an exploded view of an embodiment of the tracheal tube 230, illustrating details of the adjustable flange 234. In the depicted embodiment, the end connector 236 may be manufactured to include two circular chambers, such as a chamber 240. The flanges, such as the flange 234, may include a cylindrically-shaped component 242 suitable for being placed inside of the chamber 240. The cylindrically-shaped component 242 enables the flange 234 to move axially with respect to the end connector 236. A bottom of the chamber may act as a lower mechanical stop, thus preventing the flange 234 from extending too far backwards towards the cannula 14. Likewise, the cover 238 (shown in FIG. 18) may act as an upper mechanical stop, preventing the flange 234 from extending too far forward towards the end connector 236.

The flange 234 may also rotate radially with respect to the end connector 236. Accordingly, a tab component 244 of the flange 234 may include a curved rear section 246 suitable for following the circular contour of the exterior of the chamber 240. In order to prevent excessive radial rotation, a first end 248 of the walls of the chamber 240 may act as a mechanical stop, preventing excessive radial rotation of the flange 234 in one direction. Likewise, a second end 250 of the walls of the chamber 240 may act as a mechanical stop, preventing excessive radial rotation of the flange 234 in the other direction. An interference fit between the cylindrically-shaped component 242 and the chamber 240 may be suitable for maintaining desired flange positions. However, the user may apply an axial or a radial force sufficient to overcome the interference fit and thus manually reposition the flange 232 or 234.

In the depicted embodiment, the use of the cylindrically-shaped component 242, the cover 238, and the chamber 240 allows for a smooth movement along the X or Y axes while limiting any “twisting” motion. More specifically, the embodiment enables two degrees of freedom. One degree of freedom is enabled based on radial movements (e.g., left to right movements) about the end connector 236 and a second degree of freedom is enabled based on the axial movements (e.g., up and down movements) about the end connector 236. Any twisting motion may then be mechanically stopped by the cover 238 and/or the bottom surface and side walls of the chamber 240. In other embodiments, the cylindrically-shaped component 242, the cover 238, and the chamber 240 may be shaped to allow more degrees of motion. In yet other embodiments, other techniques such as a locking universal joint may be used to provide a full six degrees of freedom.

In certain embodiments, the flanges 232 and/or 234 may be “lockable” flanges. That is, the flanges may be securely fastened at a desired angle δ and/or α. In these embodiments, features such as openings or grooves disposed on the inside wall of the chamber 240 may be mated to protrusions such as pegs extending outwardly from the cylindrically-shaped component 242. Such pegs may be spring-driven, allowing for a secure fastening of the cylindrically-shaped component 242 to the chamber 240. Further, such a spring may compress when the user exerts a force suitable for moving flange 232 or 234 radially or axially. The spring may allow for the user to manually disengage and re-lock the flanges 232 and 234. It is to be understood that other lockable features such as teeth, radial ridges, barbs, and so forth, may be used.

Various disclosed techniques allow for the in situ reconfiguration of tracheal tube embodiments to more comformably fit a variety of patient anatomies. Indeed, the techniques disclosed herein are capable of the secure and proper placement of tracheal tube embodiments while allowing for patient neck movements. Such capabilities may result in a more efficient and comfortable airway management and ventilation.

What is claimed is:

1. A tracheal tube comprising:
an angularly adjustable first flange; and
an end connector coupled to a proximal end of the tracheal tube, wherein the angularly adjustable first flange is configured to be angularly positioned at a first angle about the end connector by a first movement of the adjustable first flange with respect to the end connector.

2. The tracheal tube of claim 1, wherein the first movement of the angularly adjustable first flange with respect to the end connector comprises a radial movement of the angularly adjustable first flange with respect to the end connector.

3. The tracheal tube of claim 1, wherein the first movement of the angularly adjustable first flange with respect to the end connector comprises an axial movement of the angularly adjustable first flange with respect to the end connector.

4. The tracheal tube of claim 1, comprising an angularly adjustable second flange, wherein the angularly adjustable second flange is configured to be positioned at a second angle about the end connector by a second movement of the adjustable second flange with respect to the end connector.

5. The tracheal tube of claim 4, wherein the first angle or the second angle is between approximately 0° to 90° relative to the end connector, or wherein both the first angle and the second angle are between approximately 0° to 90° relative to the end connector.

6. The tracheal tube of claim 1, wherein the first angle is adjustable in a plurality of discrete angular positions.

7. The tracheal tube of claim 1, comprising a non-angularly adjustable second flange.

8. The tracheal tube of claim 7, comprising an angularly adjustable flange extension, wherein the angularly adjustable flange extension is configured to be coupled to the non-angularly adjustable second flange.

9. The tracheal tube of claim 1, wherein the angularly adjustable first flange is configured to be manually angularly repositioned at a second angle about the end connector by a second movement of the angularly adjustable first flange with respect to the end connector.

10. The tracheal tube of claim 1, wherein the angularly adjustable first flange is configured to be locked at the first angle.

11. The tracheal tube of claim 10, wherein the angularly adjustable first flange is configured to be unlocked and manually angularly repositioned at a second angle about the end connector by a second movement of the angularly adjustable first flange with respect to the end connector.

12. The tracheal tube of claim 1, wherein the end connector comprises an extendable bore, and wherein the end connector is configured to be adjusted to extend axially from the end connector at a desired one of a plurality of lengths.

13. The tracheal tube of claim 1, comprising an angularly adjustable flange extension, wherein the angularly adjustable flange extension is configured to be coupled to the angularly adjustable first flange at a second angle relative to the angularly adjustable first flange.

14. The tracheal tube of claim 1, wherein the angularly adjustable first flange is configured to be adjusted to two or more degrees of freedom.

15. A tracheal tube kit comprising:
a tracheal tube;
an end connector in fluid communication with the tracheal tube;
a first angularly adjustable flange having a first length and configured to be coupled to the tracheal tube at a first angle relative to the end connector; and
a second angularly adjustable flange having a second length and configured to be coupled to the tracheal tube at a second angle relative to the end connector, wherein the first and the second flanges may be repositioned relative to the end connector.

16. The tracheal tube kit of claim 15, wherein the first length is different from the second length.

17. The tracheal tube kit of claim 15, comprising a angularly adjustable flange extension having a third length and configured to be coupled to the first or to the second flanges at a third angle relative to the first or second flanges.

18. The tracheal tube kit of claim 15, comprising a third angularly adjustable flange having a third length, and wherein the first or the second flanges may be decoupled from the tracheal tube and replaced by the third flange.

19. A tracheal tube comprising:
an angularly adjustable and lockable first flange; and
an end connector, wherein the angularly adjustable and lockable first flange is configured to be manually locked to the tracheal tube at a first angle about the end connector.

20. The tracheal tube of claim 19, wherein the angularly adjustable and lockable first flange comprises a button, and wherein the button may be used to lock and unlock the first flange.

21. The tracheal tube of claim 19, wherein the angularly adjustable and lockable first flange comprises a flexible ring, and wherein the flexible ring may be used to lock and unlock the first flange.

22. The tracheal tube of claim 19, wherein the angularly adjustable and lockable first flange comprises a plurality of teeth, and wherein the plurality of teeth may be used to lock and unlock the first flange.

23. A tracheal tube comprising:
a first flange; and
an angularly adjustable flange extension; wherein the angularly adjustable flange extension is configured to be coupled to the first flange and angularly positioned at a first angle about the first flange.

24. The tracheal tube of claim 23, wherein the first flange comprises a non-angularly adjustable first flange.

25. The tracheal tube of claim 23, wherein the angularly adjustable flange extension is configured to be manually angularly repositioned at a second angle about the first flange.
